(19) United States
(12) Patent Application Publication
(10) Pub. No.: US 2014/0069434 A1
(43) Pub. Date: Mar. 13, 2014

(54) LEVER ARM CUSHION ATTACHMENT MECHANISM

(71) Applicant: KONINKLIJKE PHILIPS N.V., EINDHOVEN (NL)

(72) Inventor: Adam Michael Neff, Oakmont, PA (US)

(73) Assignee: KONINKLIJKE PHILIPS N.V., EINDHOVEN (NL)

(21) Appl. No.: 14/117,632

(22) PCT Filed: May 16, 2012
(86) PCT No.: PCT/IB12/52450

§ 371 Date: Nov. 14, 2013

Related U.S. Application Data
(60) Provisional application No. 61/487,013, filed on May 17, 2011.

(51) Int. Cl. A61M 16/06

(52) U.S. Cl. CPC A61M 16/06
USPC 128/206.24; 29/428

(57) ABSTRACT

A patient interface device includes a frame member having a front surface and a rear surface, the frame member including a faceplate portion, the frame member having an orifice extending therethrough, and a cushion assembly including a support frame and a sealing cushion coupled to a coupling portion of the support frame. The support frame includes a lever arm extending from the coupling portion, wherein the lever arm is received through the orifice and engages an engagement surface forming a part of the front surface of the frame member, and wherein a top surface of the coupling portion engages a rear portion of the faceplate portion forming a part of the rear surface of the frame member.

### CROSS-REFERENCE TO RELATED APPLICATIONS

This patent application claims the priority benefit under 35 U.S.C. §119(e) of U.S. Provisional Application No. 61/487,013 filed on May 17, 2011, the contents of which are herein incorporated by reference.

### BACKGROUND OF THE INVENTION

1. Field of the Invention

The present invention relates to respiratory therapy systems, such as non-invasive ventilation and pressure support systems, and, in particular, to a patient interface device for a respiratory therapy system that includes a lever arm attachment mechanism for attaching a facial sealing cushion to a frame member.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible cushion on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. Such patient interface devices may also employ other patient contacting components, such as forehead supports, cheek pads and chin pads. The patient interface device is connected to a gas delivery tube or conduit and interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

Adherence and compliance to therapy, such as CPAP or other pressure support therapies, is growing to be an industry-wide issue. Factors such as comfort and ease of a patient interface device can greatly affect a patient's adherence and compliance to therapy.

Current patient interface devices having removable cushions typically employ one of three main types of attachment mechanisms for attaching the cushion to the faceplate thereof. The most common mechanism employs a physical snap on each side of the cushion (e.g., most commonly each of the three sides of a triangular cushion) that applies a force in the center of each side to maintain a proper seal. Another mechanism employs a hinge and two snaps, one on each side of the cushion. In these implementations, one of the multiple snaps is often missed, which causes a mask leak. The third mechanism employs a groove in the faceplate into which the cushion is pressed. The cushion, when inserted into the groove, presses against the two sides of the groove to create a proper seal. Insertion and removal of the cushion in this implementation can be difficult (the patient must ensure that the cushion is seated all of the way around), and does not provide an audible snapping sound to let the patient know that the cushion has been properly attached.

### SUMMARY OF THE INVENTION

In one embodiment, a patient interface device is provided that includes a frame member having a front surface and a rear surface opposite the front surface, the frame member including a faceplate portion, the frame member having an orifice extending from the front surface of the frame member to the rear surface of the frame member, and a cushion assembly including a support frame and a sealing cushion coupled to a coupling portion of the support frame. The support frame includes a lever arm extending from the coupling portion, wherein the lever arm is received through the orifice and engages an engagement surface forming a part of the front surface of the frame member, and wherein a top surface of the coupling portion engages a rear portion of the faceplate portion forming a part of the rear surface of the frame member.

In another embodiment, a method of assembling a patient interface device is provided that includes holding a frame member having a front surface and a rear surface opposite the front surface, the frame member including a faceplate portion, the frame member having an orifice extending from the front surface of the frame member to the rear surface of the frame member, and holding a cushion assembly including a support frame and a sealing cushion coupled to a coupling portion of the support frame, the support frame having a lever arm extending from the coupling portion. The method further includes inserting the lever arm through the orifice, and rotating the cushion assembly relative to the frame member and causing the lever arm to engage an engagement surface forming a part of the front surface of the frame member and a top surface of the coupling portion to engage a rear portion of the faceplate portion forming a part of the rear surface of the frame member.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of a system adapted to provide a regimen of respiratory therapy to a patient according to one exemplary embodiment of the present invention;

FIG. 2 is a front isometric view and FIG. 3 is a rear elevational view of a frame member forming a part of a patient interface device of the system of FIG. 1;

FIG. 4 is an isometric view of a cushion assembly forming a part of a patient interface device of the system of FIG. 1;

FIG. 5 is an isometric view and FIG. 6 is a front elevational view of a support frame forming a part of the cushion assembly id FIGS. 4; and

FIG. 7 is an isometric view showing the manner in which the cushion assembly of FIG. 4 is attached to the frame member of FIGS. 2 and 3.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

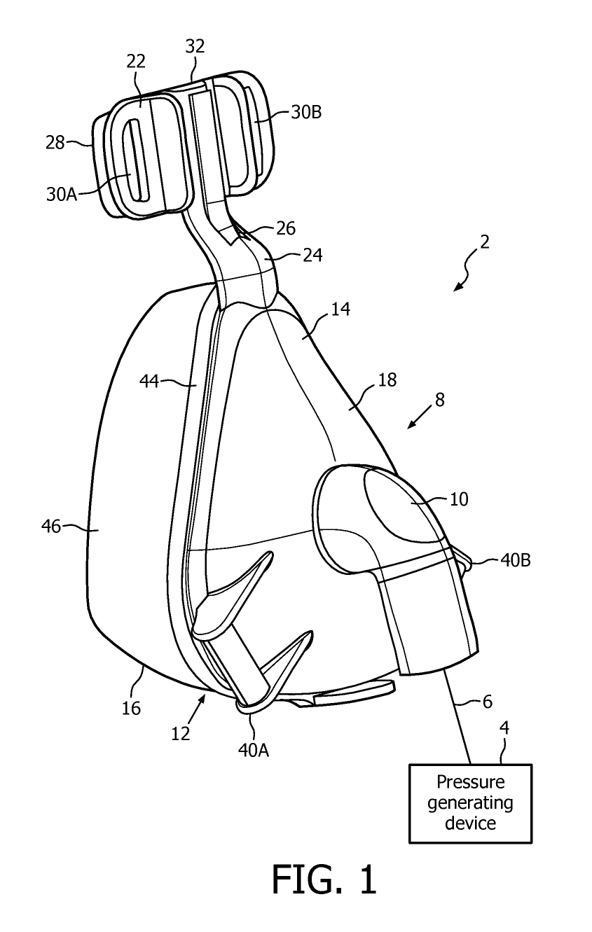

A system 2 adapted to provide a regimen of respiratory therapy to a patient according to one exemplary embodiment is generally shown in FIG. 1. System 2 includes a pressure generating device 4, a patient circuit 6, and a patient interface device 8 having a fluid coupling conduit 10. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex® or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8 through fluid coupling conduit 10, which in the illustrated embodiment is an elbow connector. Delivery conduit 6 and patient interface device 8 are often collectively referred to as a patient circuit.

In the exemplary embodiment, patient interface 8 includes a patient sealing assembly 12, which in the illustrated embodiment is a nasal/oral mask. However, other types of patient sealing assemblies, such as, without limitation, a nasal mask or a full face mask, which facilitates the delivery of the flow of breathing gas to the airway of a patient, may be substituted for patient sealing assembly 12 while remaining within the scope of the present invention. Patient sealing assembly 12 includes a frame member 14 having a cushion assembly 16 coupled thereto, each of which is described in greater detail below.

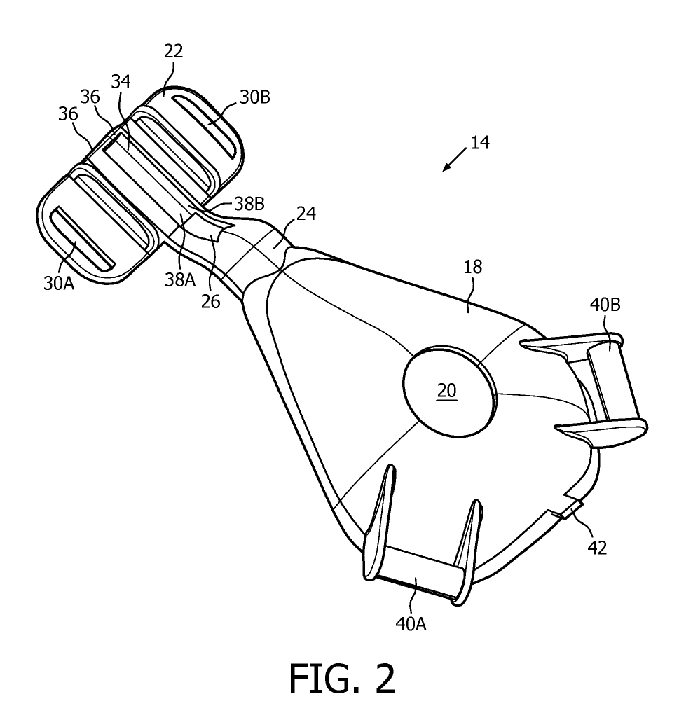

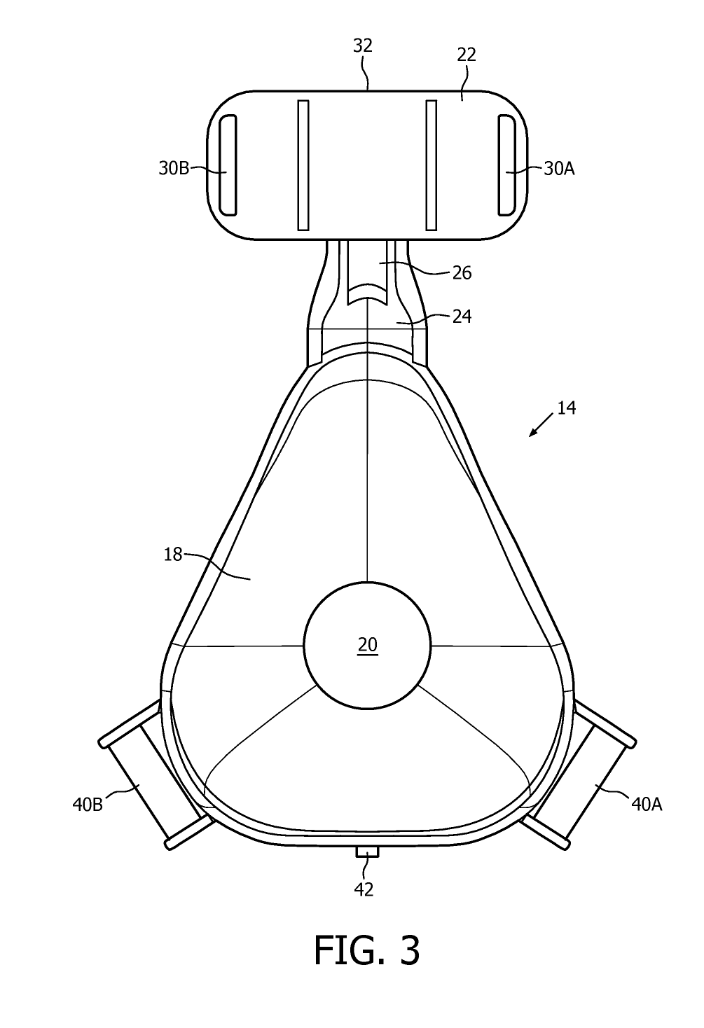

FIG. 2 is a front isometric view and FIG. 3 is a rear elevational view of frame member 14 according to the exemplary embodiment. In the illustrated embodiment, frame member 14 is made of a rigid or semi-rigid material, such as, without limitation, an injection molded thermoplastic or silicone, and includes a faceplate portion 18 having an opening 20. As seen in FIG. 1, fluid coupling conduit 10 is coupled to faceplate portion 18 through opening 20, which configuration allows the flow of breathing gas from pressure generating device 4 to be communicated to an interior space defined by cushion assembly 16, and then to the airway of a patient.

Frame member 14 also includes a forehead support member 22 that is coupled to faceplate portion 18 by a connecting member 24. An orifice 26 extends through connecting member 24, the function of which is described elsewhere herein. As seen in FIG. 1, a forehead cushion 28 is coupled to the rear of forehead support member 22. In the exemplary embodiment, forehead cushion 28 is made of a unitary piece of soft, flexible, cushiony, elastomeric material, such as, without limitation, silicone, an appropriately soft thermoplastic elastomer, a closed cell foam, or any combination of such materials. In addition, forehead support member 22 includes looped connectors 30A, 30B to which the upper straps of a headgear component (not shown) may be attached. Furthermore, the central portion 32 of forehead support member 22 includes a receiving channel 34 adjacent to orifice 26 defined by a top wall 36 and side walls 38A, 38B.

Connecting elements 40A, 40B are attached to the bottom of faceplate portion 18, and are structured to receive and hold the upper straps of a headgear component (not shown). In addition, a peg member 42 extends outwardly from the bottom of faceplate portion. The function of peg member 42 is described below.

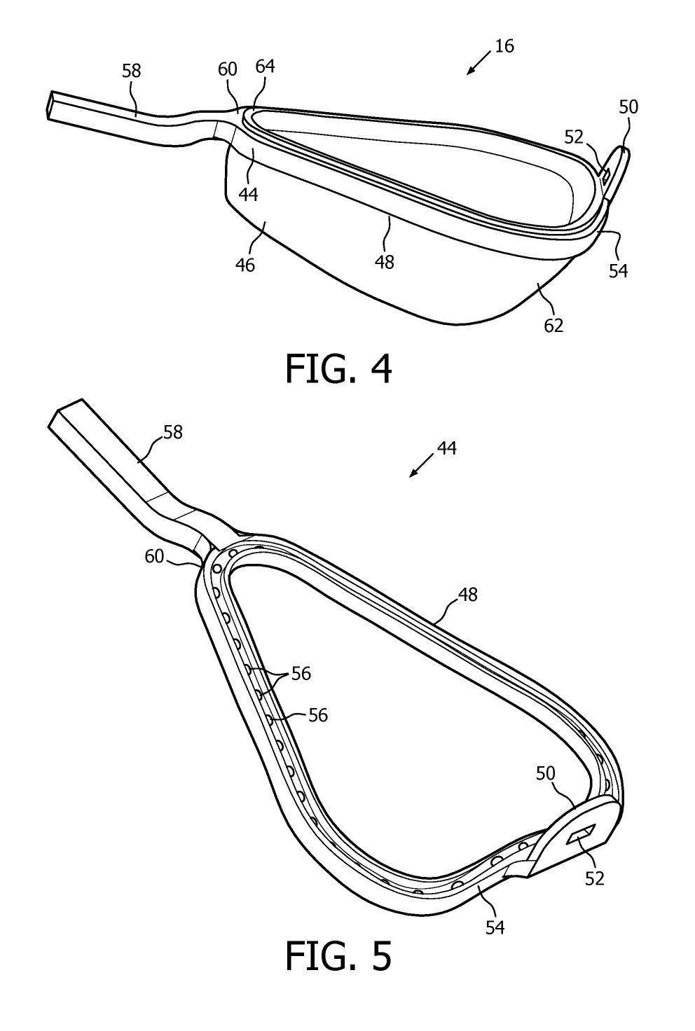

FIG. 4 is an isometric view of cushion assembly 16. Cushion assembly 16 includes a support frame 44 and a sealing cushion 46 coupled to support frame 44. In the illustrated embodiment, support frame 44 is made of a rigid or semi-rigid material, such as, without limitation, an injection molded thermoplastic or silicone, and sealing cushion 46 is defined from a unitary piece of soft, flexible, cushiony, elastomeric material, such as, without limitation, silicone, an appropriately soft thermoplastic elastomer, a closed cell foam, or any combination of such materials.

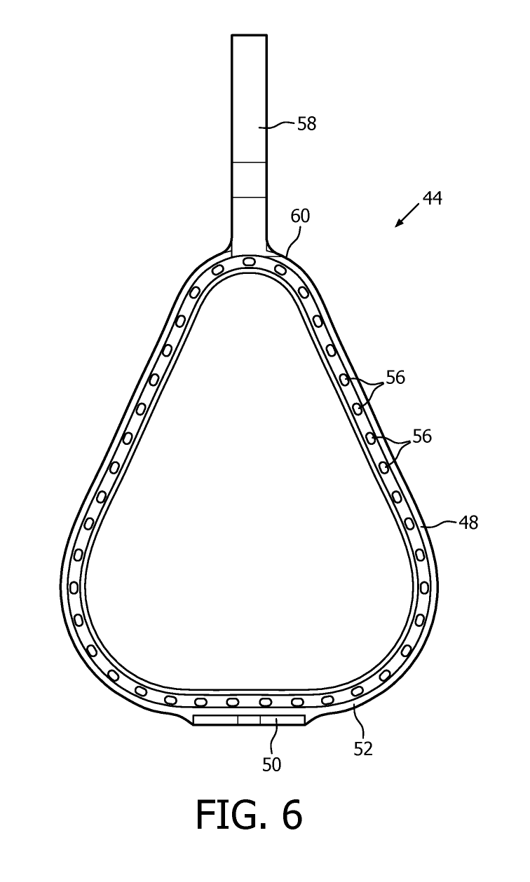

FIG. 5 is an isometric view and FIG. 6 is a front elevational view of support frame 44 according to the exemplary embodiment. Support frame 44 includes a generally triangular ring portion 48. A tab member 50 having a hole 52 provided therein is provided on the bottom end 54 of ring portion 48. Ring portion 48 also includes a plurality of holes 56 extending the through, the function of which is described below. A lever arm 58 extends from the apex 60 of ring portion 48. As seen in FIG. 4, sealing cushion 46 is coupled to ring portion 48. In the illustrated embodiment, sealing cushion 46 is overmolded onto ring portion 48 (through holes 56) such that a first portion 62 of sealing cushion 46, adapted to engage the face of the patient, extends from the bottom surface of ring portion 48 and a second portion 64 of sealing cushion 46 extends from the top surface of ring portion 48 around the outer periphery of ring portion 48.

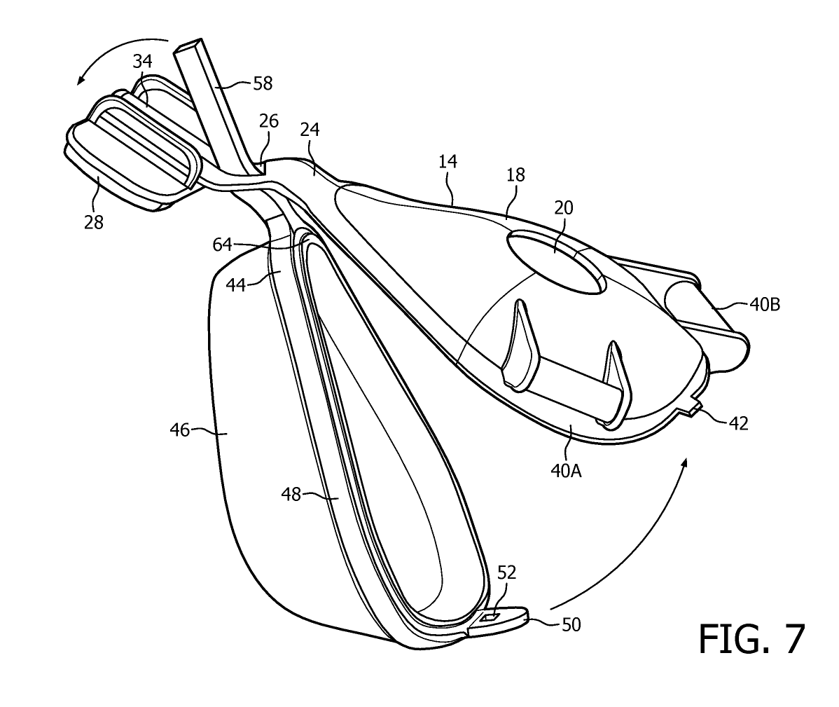

Cushion assembly 16 is removably attached to frame member 14 in the following manner. First, lever arm 58 of support frame 44 is inserted through orifice 26 of connecting member 24 of frame member 14. When this is done, the bottom of lever arm 58 will engage the front of receiving channel 34, which acts a pivot point for support frame 44. A force is then applied to lever arm 58 to cause cushion assembly 16 to rotate about the pivot point as shown by the arrows in FIG. 7. Sufficient force is applied to cause cushion assembly 16 to rotate to an extent that lever arm 58 is received in receiving channel 34 and engages the bottom surface thereof (FIG. 1). In addition, when this happens, peg member 42 is received in hole 52 of tab member 50 to secure cushion assembly 16 is place against frame member 14. When so secured, second portion 64 of sealing cushion 46 engages the outer edge of faceplate portion 18 and is compressed between ring portion 48 and faceplate portion 18 to create an airtight seal.

The insertion of peg member 42 into hole 52 produces a single audible click that indicates to the patient that the cushion assembly 16 has been properly attached to frame member 14. Receiving channel 34 may be sized to provide a friction fit between it and lever arm 58 to help secure cushion assembly 16 to frame member 14. Also, a releasable attachment mechanism, such as a detent or the like, may be provided on lever arm 58 or in receiving channel 34 to temporarily secure the two parts to one another. Cushion assembly 16 may be removed from frame member 14 by disengaging peg member 42 from hole 52 and rotating cushion assembly in the opposite direction. While peg member 42 an hole 52 are shown as the exemplary locking mechanism, it will be appreciated that other suitable locking mechanisms, such as conventional snaps or cooperating magnets, may also be used.

In alternative embodiments, the surface that lever arm 58 engages does not need to be on the forehead support member 22. Instead, it may be on other portions of the frame member 14, such as, without limitation, the connecting member 24 or the faceplate portion 18. In such embodiments, the receiving channel 34 may be provided on that engagement surface.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device, comprising:
a frame member having a front surface and a rear surface opposite the front surface, the frame member including a faceplate portion, the frame member having an orifice extending from the front surface of the frame member to the rear surface of the frame member; and
a cushion assembly including a support frame and a sealing cushion coupled to a coupling portion of the support frame, the support frame including a lever arm extending from the coupling portion, wherein the lever arm is structured to be received through the orifice and engage and be rotatable about a part of an engagement surface adjacent the orifice and forming a part of the front surface of the frame member so that a bottom surface of the lever arm is able to engage the engagement surface, and wherein a top surface of the coupling portion is structured to engage a rear portion of the faceplate portion forming a part of the rear surface of the frame member.

2. The patient interface device according to claim 1, wherein the frame member includes a forehead support member and a connecting member extending between the faceplate portion and the forehead support member, wherein the orifice is provided in the connecting member, and wherein the engagement surface comprises a front portion of the forehead support member.

3. (canceled)

4. The patient interface device according to claim 2, wherein the front portion of the forehead support member comprises a receiving channel provided on the forehead support member, and wherein the lever arm is structured to engage and be rotatable about a front of the receiving channel.

5. The patient interface device according to claim 4, wherein the receiving channel is defined by a top wall and first and second side walls provided on the forehead support member.

6. The patient interface device according to claim 1, wherein at least a first part the coupling portion is releasably attached to a first part of the faceplate portion.

7. The patient interface device according to claim 6, wherein the first part the coupling portion includes a first components of a releasable attachment mechanism and the first part of the faceplate portion includes a second component of the releasable attachment mechanism structured to be releasably attached to the first component.

8. The patient interface device according to claim 7, wherein the first component comprises a member having a hole provided therein, and the second component comprises a peg member structured to be received in the hole.

9. The patient interface device according to claim 1, wherein a first engagement portion of the sealing cushion is adapted to engage a face of a patient and extends from a bottom surface of the coupling portion, wherein a second engagement portion of the sealing cushion extends from the top surface of coupling portion around an outer periphery of the coupling portion, and wherein the second engagement portion is sandwiched in between the top surface of the coupling portion and the rear portion of the faceplate portion.

10. A method of assembling a patient interface device;
holding a frame member having a front surface and a rear surface opposite the front surface, the frame member including a faceplate portion, the frame member having an orifice extending from the front surface of the frame member to the rear surface of the frame member;
holding a cushion assembly including a support frame and a sealing cushion coupled to a coupling portion of the support frame, the support frame having a lever arm extending from the coupling portion;
inserting the lever arm through the orifice; and
rotating the cushion assembly relative to the frame member and causing the lever arm to engage an engagement surface forming a part of the front surface of the frame member and causing a top surface of the coupling portion to engage a rear portion of the faceplate portion forming a part of the rear surface of the frame member.

11. The method according to claim 10, wherein the frame member includes a forehead support member and a connecting member extending between the faceplate portion and the forehead support member, wherein the orifice is provided in the connecting member, and wherein the engagement surface comprises a front portion of the forehead support member.

12. The method according to claim 11, wherein the front portion of the forehead support member comprises a receiving channel provided on the forehead support member, and wherein the rotating causes the lever arm to be received within the receiving channel.

13. The method according to claim 12, wherein the receiving channel is defined by a top wall and first and second side walls provided on the forehead support member.

14. The method according to claim 10, further comprising releasably attaching a first part the coupling portion to a first part of the faceplate portion.

15. The method according to claim 10, wherein a first engagement portion of the sealing cushion is adapted to engage a face of a patient and extends from a bottom surface of the coupling portion, wherein a second engagement portion of the sealing cushion extends from the top surface of coupling portion around an outer periphery of the coupling portion, and wherein during the rotating the second engagement portion is sandwiched in between the top surface of the coupling portion and the rear portion of the faceplate portion in a manner that creates a seal between cushion assembly and the frame member.
